(19) United States
(12) Patent Application Publication
(10) Pub. No.: US 2022/0379087 A1
(43) Pub. Date: Dec. 1, 2022

(54) Catheter Extension Set Having a Patency or Monitoring Instrument

(71) Applicant: Becton, Dickinson and Company, Franklin Lakes, NJ (US)

(72) Inventors: Jonathan Karl Burkholz, Salt Lake City, UT (US); Megan Scherich, Salt Lake City, UT (US)

(21) Appl. No.: 17/752,312

(22) Filed: May 24, 2022

Related U.S. Application Data
(60) Provisional application No. 63/193,489, filed on May 26, 2021.

(51) Int. Cl. A61M 25/00; A61B 5/15; A61B 5/154; A61M 25/01

(52) U.S. Cl. CPC A61M 25/0084; A61B 5/150992; A61B 5/154; A61M 25/0136; A61M 2025/0095

(57) ABSTRACT

A catheter extension set may include a housing, which may include a distal end, a proximal end, and a lumen. The catheter extension set may include a distal connector coupled to the distal end of the housing and configured to couple to a catheter assembly. The catheter extension set may include a handle, an advancement element, and an instrument. The instrument may extend through a U-shaped channel of the advancement element and may include a fixed first end and a second end. A translation feature may be disposed between the handle and the housing and within a pocket of the advancement element such that in response to distal movement of the handle a first distance, the advancement element moves distally within the lumen the first distance and the second end of the instrument advances distally a second distance. The second distance may be at least twice the first distance.

### CROSS-REFERENCE TO RELATED APPLICATION

The present application claims priority to U.S. Provisional Application Ser. No. 63/193,489, entitled “Catheter Extension Set Having a Patency or Monitoring Instrument”, filed May 26, 2021, the entire disclosure of which is hereby incorporated by reference in its' entirety.

### BACKGROUND OF THE INVENTION

Catheters are commonly used for a variety of infusion therapies. For example, catheters may be used for infusing fluids, such as normal saline solution, various medicaments, and total parenteral nutrition, into a patient. Catheters may also be used for withdrawing blood from the patient.

A common type of catheter device includes a catheter that is over-the-needle. As its name implies, the catheter that is over-the-needle may be mounted over an introducer needle having a sharp distal tip. A catheter assembly may include a catheter adapter, the catheter extending distally from the catheter adapter, and the introducer needle extending through the catheter. The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into vasculature of the patient.

In order to verify proper placement of the introducer needle and/or the catheter in the blood vessel, a clinician generally confirms that there is “flashback” of blood in a flashback chamber of the catheter assembly. Once placement of the needle has been confirmed, the clinician may temporarily occlude flow in the vasculature and remove the needle, leaving the catheter in place for future blood withdrawal or fluid infusion.

Infusion and blood withdrawal using the catheter may be difficult for several reasons, particularly when an indwelling time of the catheter increase. A fibrin sheath or thrombus may form on an internal surface of the catheter assembly, an external surface of the catheter assembly, or within the vasculature near the distal tip of the catheter. The fibrin sheath or thrombus may block or narrow a fluid pathway through the catheter, which may impair infusion and/or collection of a high-quality blood sample.

The subject matter claimed herein is not limited to embodiments that solve any disadvantages or that operate only in environments such as those described above. Rather, this background is only provided to illustrate one example technology area where some implementations described herein may be practiced.

### SUMMARY OF THE INVENTION

The present disclosure relates generally to vascular access devices and related methods. In particular, the present disclosure relates to a catheter extension set, as well as related devices, systems, and methods. In some embodiments, a catheter extension set may include a housing, which may include a distal end, a proximal end, and a lumen extending through the distal end of the housing. In some embodiments, the catheter extension set may include a flexible extension tube.

In some embodiments, the catheter extension set may include a distal connector, which may be coupled to the distal end of the housing and configured to couple to a catheter assembly. In some embodiments, the catheter extension set may include a handle. In some embodiments, the handle may not contact the housing. In some embodiments, the catheter extension set may include an advancement element, which may include a U-shaped channel. In some embodiments, the advancement element may include a pocket. In some embodiments, the advancement element may be disposed within the lumen.

In some embodiments, the catheter extension set may include an instrument extending through the U-shaped channel. In some embodiments, the instrument may include a fixed first end and a second end configured to advance distally a first distance in response to distal movement of the handle a second distance. In some embodiments, the first distance may be at least twice the second distance.

In some embodiments, the catheter extension set may include a translation feature disposed between the handle and an outer surface of the housing and within the pocket such that in response to distal movement of the handle along the outer surface of the housing the first distance, the advancement element moves distally within the lumen the first distance, and the second end of the instrument advances distally the second distance.

In some embodiments, the housing may include another lumen. In some embodiments, the other lumen may extend through the distal end of the housing and the proximal end of the housing and may be configured for fluid flow therethrough. In some embodiments, the catheter extension set may include an elastomeric seal, which may be disposed within the distal connector or a distal end of the housing. In some embodiments, the instrument may be configured to extend through the elastomeric seal. In some embodiments, the elastomeric seal may be positioned to allow distal fluid flow into the other lumen but not the lumen.

In some embodiments, the catheter extension set may include a blood collection device in fluid communication with the distal connector and the other lumen. In some embodiments, the blood collection device may include a syringe or a sharp cannula configured to receive an evacuated blood collection tube. In some embodiments, the catheter extension set may include a proximal connector coupled to a proximal end of the housing. In some embodiments, the blood collection device may be coupled to the proximal connector.

In some embodiments, the handle may include a gap. In some embodiments, the housing the other lumen may extend through the distal end of the housing and the proximal end of the housing. In some embodiments, the other lumen may be aligned with the gap. In some embodiments, the other lumen may be disposed on top of the lumen.

In some embodiments, a proximal end of the lumen may be closed. In some embodiments, the catheter extension set may include an additional translation feature disposed on an opposite side of the housing from the translation feature and between the housing and the handle.

In some embodiments, the catheter extension set may include a port and an extension tube coupled to the port. In some embodiments, the port may be disposed distal to the elastomeric seal and configured for fluid flow therethrough. In some embodiments, the blood collection device may be coupled to the extension tube.

In some embodiments, the catheter extension set may include a grip feature proximate and proximal to the handle when the instrument is in a fully retracted position. In some embodiments, an outer surface of the grip feature may be textured.

In some embodiments, the catheter extension set may include a pair of opposing translation features disposed between the handle and an outer surface of the housing. In some embodiments, the pair of opposing translation features may be disposed proximal to the advancement element and configured to push the advancement element distally. In some embodiments, in response to distal movement of the handle along the outer surface of the housing the first distance, the pair of opposing translation features may push the advancement element distally within the lumen the first distance, and the second end of the instrument may advance distally the second distance.

In some embodiments, the catheter extension set may include another pair of opposing translation features disposed between the handle and an outer surface of the housing. In some embodiments, the other pair of opposing translation features may be disposed distal to the advancement element and are configured to push the advancement element proximally in response to proximal movement of the handle.

In some embodiments, a method of blood collection may include inserting a catheter of the catheter assembly into vasculature of a patient. In some embodiments, the method may include coupling the catheter extension set to the catheter assembly. In some embodiments, the method may include moving the handle distally the second distance. In some embodiments, in response to moving the handle distally the second distance, the second end of the instrument may advance into or through the catheter.

In some embodiments, the housing may include the other lumen, which may extend through the distal end of the housing and the proximal end of the housing and may be configured for fluid flow therethrough. In some embodiments, the method may include moving the handle proximally the second distance. In some embodiments, in response to moving the handle proximally the second distance, the second end of the instrument may be retracted into the catheter extension set. In some embodiments, the method may include collecting blood through the other lumen of the housing.

It is to be understood that both the foregoing general description and the following detailed description are examples and explanatory and are not restrictive of the invention, as claimed. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE DRAWINGS

Example embodiments will be described and explained with additional specificity and detail through the use of the accompanying drawings in which:

FIG. 1A is a top view of an example catheter extension set, illustrating an example handle in a proximal position, according to some embodiments;

FIG. 1B is a top view of the catheter extension set of FIG. 1A, illustrating the handle in a distal position, according to some embodiments;

FIG. 1C is a side view of the catheter extension set of FIG. 1A, illustrating the handle in the distal position, according to some embodiments;

FIG. 1D is a cross-sectional view of the catheter extension set of FIG. 1A along the line 1D-1D of FIG. 1A, according to some embodiments;

FIG. 1E is a cross-sectional view of the catheter extension set of FIG. 1A along the line 1E-1E of FIG. 1D, according to some embodiments;

FIG. 1F is a cross-sectional view of the catheter extension set of FIG. 1A along the line 1F-1F of FIG. 1D, according to some embodiments;

FIG. 1G is a cross-sectional view of the catheter extension set of FIG. 1A along the line 1G-1G of FIG. 1C, according to some embodiments;

FIG. 1H is a cross-sectional view of the catheter extension set of FIG. 1A, according to some embodiments;

FIG. 2A is a cross-sectional side view of an example catheter extension set, according to some embodiments;

FIG. 2B is a cross-sectional top view of the catheter extension set of FIG. 2A, according to some embodiments;

FIG. 3 is a top view of the catheter extension set of FIG. 1A, illustrating an example grip element, according to some embodiments;

FIG. 4 is a top view of the catheter extension set of FIG. 1A, illustrating an example port, according to some embodiments;

FIG. 5 is a top view of the catheter extension set of FIG. 1A coupled to an example catheter assembly, according to some embodiments;

FIG. 6A is a top view of an example catheter extension set, according to some embodiments;

FIG. 6B is a cross-sectional view of a portion of the catheter extension set of FIG. 6A, according to some embodiments;

FIG. 6C is a cross-sectional view of another portion of the catheter extension set of FIG. 6A, according to some embodiments

FIG. 7A is an upper perspective view of an example housing, according to some embodiments;

FIG. 7B is an upper perspective view of the housing, according to some embodiments; and

FIG. 7C is an upper perspective view of the housing, according to some embodiments.

### DETAILED DESCRIPTION OF THE INVENTION

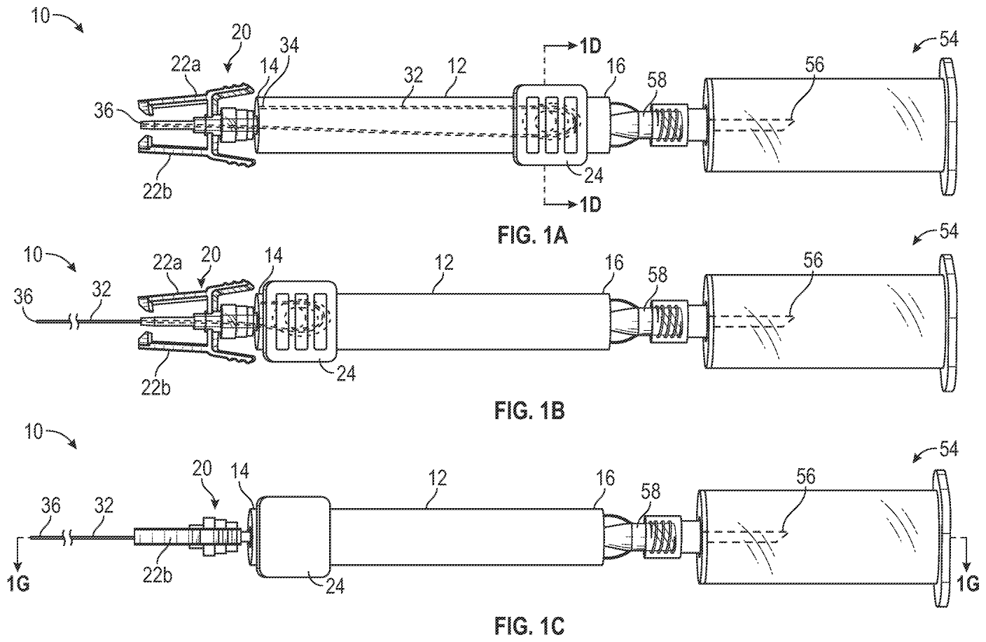

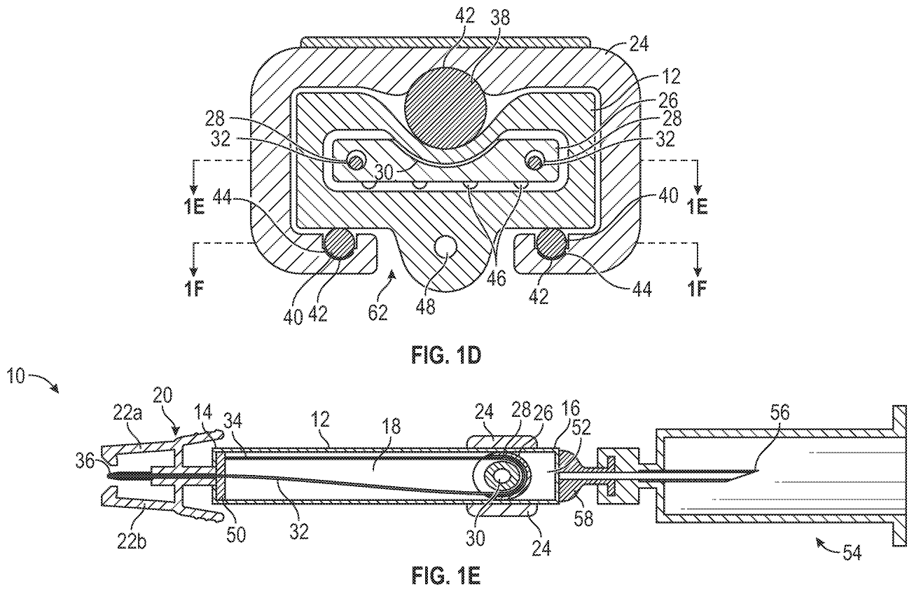

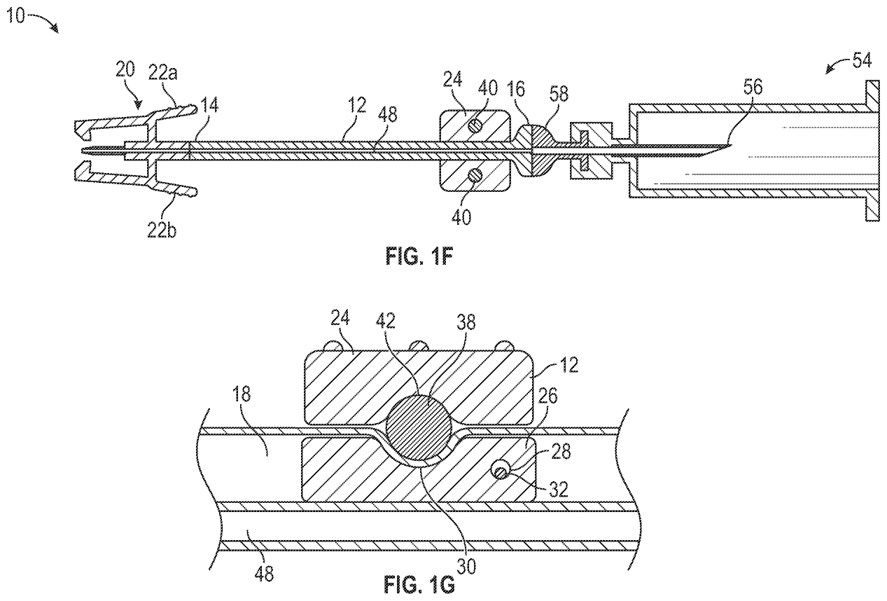

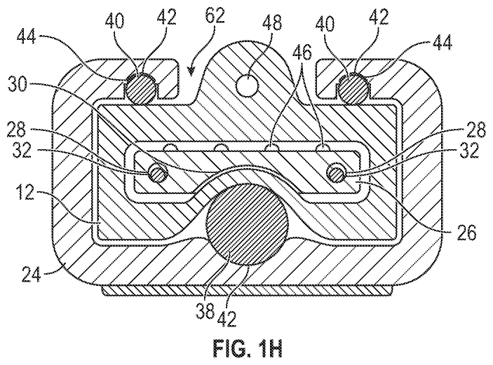

Referring now to FIGS. 1A-1G, in some embodiments, a catheter extension set 10 may include a housing 12, which may include a distal end 14, a proximal end 16, and a lumen 18 extending through the distal end 14 of the housing 12. In some embodiments, the catheter extension set 10 may include a distal connector 20, which may be coupled to the distal end 14 of the housing 12 and configured to couple to a catheter assembly.

In some embodiments, the distal connector 20 may include opposing lever arms 22a,22b. In some embodiments, distal ends of the opposing lever arms 22a,22b may be configured to move apart from each other in response to pressure applied to proximal ends of the opposing lever arms 22a,22b. In some embodiments, in response to removal of the pressure applied to the proximal ends of the opposing lever arms 22a,22b, the distal ends may move closer to each other and clasp a portion of the catheter assembly, such as a needleless connector, another connector, or a proximal end of a catheter adapter, for example. In some embodiments, the distal connector 20 may include a blunt cannula or male luer configured to insert into the portion of the catheter assembly. In some embodiments, the distal connector 20 may include another suitable connector. For example, the distal connector 20 may include a threaded male luer, a slip male luer, a threaded male luer with a spin lock, a threaded male luer with a removable blunt cannula snap connection, a slip male luer with a removable blunt cannula snap connection, or another suitable connector. In some embodiments, the distal connector 20 may include one or more bond pockets each configured to receive an extension tube.

In some embodiments, the catheter extension set 10 may include a handle 24, which may be configured for bi-directional movement to advance and retract the instrument 32. In some embodiments, an outer surface or top surface of the handle 24 may include one or more protrusions, which may facilitate gripping of the handle 24 by a user. In some embodiments, the catheter extension set 10 may include an advancement element 26, which may include a U-shaped channel 28. In some embodiments, the advancement element 26 may include a depression or pocket 30. In some embodiments, the pocket 30 may be disposed interior of the U-shaped channel 28. In some embodiments, the advancement element 26 may be disposed within the lumen 18.

In some embodiments, the catheter extension set 10 may include an instrument 32 extending through the U-shaped channel 28. In some embodiments, the instrument 32 may include a guidewire, a probe, a guidewire or a probe with one or more sensors, or another suitable instrument. In some embodiments, the sensors may be used for patient or device monitoring and may include sensors mearing pressure, temperature, pH, blood chemistry, oxygen saturation, flow rate, or another physiological measure. In some embodiments, the instrument 32 may improve patency of a catheter of the catheter assembly for medication and fluid delivery, as well as blood acquisition, through a dwell time of the catheter. In some embodiments, the catheter may be indwelling or inserted in vasculature of a patient when the catheter extension set 10 is coupled to the catheter assembly. In some embodiments, the catheter extension set 10 may facilitate blood flow and quality during blood collection, improve workflow, and reduce a risk of manipulation-related catheter complications.

In some embodiments, the instrument 32 may include a fixed first end 34 and a second end 36 configured to advance distally and/or be retracted proximally. In some embodiments, the second end 36 may include a rod, a coil, or any other suitable shape configured to facilitate thrombus removal and/or decrease damage to the vasculature. In some embodiments, the coil may surround the rod. In some embodiments, a distalmost portion of the second end 36 may be blunt to decrease a risk of damaging the vasculature. In some embodiments, the second end 36 may be porous to facilitate blood flow therethrough. In some embodiments, the fixed first end 34 may be fixed inside the housing 12. For example, the fixed first end 34 may be adhered, bonded, or secured within the housing 12 in another suitable manner.

In some embodiments, the second end 36 may be configured to advance distally a first distance in response to distal movement of the handle 24 a second distance. In some embodiments, the first distance may be at least twice the second distance. In some embodiments, movement of the instrument 32 may be facilitated without contacting the instrument 32 with the handle 24, which may reduce a risk of bacterial contamination of the instrument 32.

In some embodiments, the catheter extension set 10 may include a translation feature 38 disposed between the handle 24 and an outer surface of the housing 12 and within the pocket 30 such that in response to distal movement of the handle 24 along the outer surface of the housing 12 the first distance, the advancement element 26 may move distally within the lumen 18 the first distance, and the second end 36 of the instrument 32 may advance distally the second distance. In some embodiments, the translation feature 38 may include a ball, a wheel, a roller bearing, or another suitable feature configured to create a localized compression of the housing 12.

In some embodiments, the housing 12 may be constructed of a flexible material, such as thermoplastic elastomer (TPE), thermoplastic polyurethane (TPU), PELLETHANE®, polyvinyl chloride (PVC), or another suitable material. In some embodiments, the flexible material may facilitate compression of the housing 12 by the translation feature 38. In some embodiments, the housing 12 may include a flexible extension tube. In some embodiments, the housing 12 may be constructed of one or more of the flexible material, a rigid material, and a semi-rigid material. In some embodiments, the housing 12 may be transparent to facilitate visualization of components therein and/or markings on the components, such as, for example, the instrument 32 and/or the advancement element 26.

In some embodiments, the translation feature 38 may compress the housing 12 such that the housing 12 extends into the pocket 30. In some embodiments, the translation feature 38 compressing the housing 12 into the pocket 30 may facilitate movement of the advancement element 26 with the handle 24.

In some embodiments, the catheter extension set 10 may include one or more additional translation features 40. In some embodiments, the additional translation features 40 may be disposed on an opposite side of the housing 12 from the translation feature 38 and disposed between the housing 12 and the handle 24. In some embodiments, the translation feature 38 and/or the additional translation features 40 may prevent the handle 24 from contacting the housing 12, allowing the handle 24 to move along a longitudinal axis of the housing 12 with decreased friction. In some embodiments, the additional translation features 40 may each include a roller ball, a wheel, a bearing, or another suitable feature.

In some embodiments, the translation feature 38 and/or the additional translation features 40 may be disposed within depressions 42 in the handle 24. In some embodiments, a surface of one or more of the depressions 42 may include one or more small protrusions 44, which may decrease a size of a surface area of the handle 24 in contact with the translation feature 38 and/or the additional translation features 40. In some embodiments, the depressions 42 may be shaped generally similar to the translation feature 38 and/or the additional translation features 40, which may be spherical. In some embodiments, a surface of the advancement element 26 opposite the pocket 30 may include one or more bumps 46, which may decrease a size of a surface area of the advancement element 26 in contact with the housing 12 and allow the advancement element 26 to move with respect to the housing 12 with decreased friction.

In some embodiments, the housing 12 may include another lumen 48. In some embodiments, the other lumen 48 may extend through the distal end 14 of the housing 12 and the proximal end 16 of the housing 12 and may be configured for fluid flow therethrough. In some embodiments, a width and/or length of the other lumen 48 may be configured to provide a blood flow rate that reduces blood collection time while also reducing hemolysis during blood collection. In some embodiments, the 2× or greater than 2× advancement of the instrument 32 in response to 1× advancement of the handle 24 may enable the housing 12 and the other lumen 48, used for blood collection, to be shorter, resulting in less blood volume in the other lumen 48.

In some embodiments, the catheter extension set 10 may include an elastomeric seal 50, which may be disposed within the distal connector 20 or the distal end 14 of the housing 12. In some embodiments, the instrument 32 may be configured to extend through the elastomeric seal 50. In some embodiments, the elastomeric seal 50 may be positioned to allow distal fluid flow into the other lumen 48 but may prevent distal fluid flow into the lumen 18. In some embodiments, a proximal end 52 of the lumen 18 may be closed. For example, a septum or seal may be disposed within the proximal end 52 of the lumen 18 or may be closed by the housing 12, as illustrated, for example, in FIG. 1E.

In some embodiments, the instrument 32 may be fully contained within the lumen 18. In further detail, in some embodiments, the second end 36 of the instrument 32 may be disposed proximal to the elastomeric seal 50 when the instrument 32 is in a fully retracted position and/or the handle 24 is in the proximal position. In other embodiments, the instrument 32 may extend through the elastomeric seal 50 when the instrument 32 is in the fully retracted position and/or the handle 24 is in the proximal position, but the second end 36 may be disposed within the distal connector 20.

In some embodiments, the catheter extension set 10 may include a blood collection device 54 in fluid communication with the distal connector 20 and the other lumen 48. In some embodiments, the blood collection device 54 may include a syringe or a sharp cannula 56 configured to receive an evacuated blood collection tube. In some embodiments, the sharp cannula 56 may be disposed within a holder 59 configured to hold the evacuated blood collection tube. In some embodiments, the blood collection device 54 may include a BD VACUTAINER® LUER-LOK™ access device, available from Becton Dickinson & Company of Franklin Lakes, N.J., or similar device. In some embodiments, the evacuated blood collection tube may include a BD VACUTAINER® Blood Collection Tube, available from Becton Dickinson & Company of Franklin Lakes, N.J..

In some embodiments, the catheter extension set 10 may include a proximal connector 58 coupled to the proximal end 16 of the housing 12. In some embodiments, the blood collection device 54 may be coupled to the proximal connector 58. In these and other embodiments, the proximal connector 58 may include a female luer. In some embodiments, the blood collection device 54 may be monolithically formed as a single unit with the proximal end 16 of the housing 12. In some embodiments, a needleless connector may be disposed between the proximal connector 58 and the blood collection device 54. In some embodiments, the proximal connector 58 may be coupled to an IV line or another fluidic connection to provide infusion of fluid to the vasculature of the patient through the other lumen 48.

In some embodiments, a method of blood collection may include inserting the catheter of the catheter assembly into the vasculature of the patient. In some embodiments, the method may include coupling the catheter extension set 10 to the catheter assembly. In some embodiments, the catheter extension set 10 may be coupled to the catheter assembly after the catheter is inserted into the vasculature of the patient. In some embodiments, the method may include moving the handle 24 distally the first distance. In some embodiments, in response to moving the handle distally the second distance, the second end of the instrument 32 may advance into or through the catheter.

In some embodiments, the housing 12 may include the other lumen 48, which may extend through the distal end 14 of the housing 12 and the proximal end 16 of the housing 12 and configured for fluid flow therethrough. In some embodiments, the method may include moving the handle 24 proximally the first distance. In some embodiments, the in response to moving the handle 24 proximally the first distance, the second end 36 of the instrument 32 may be retracted into the catheter extension set 10, such as, for example, entirely retracted into the catheter extension set 10. In some embodiments, the method may include collecting blood through the other lumen 48 of the housing 12.

In some embodiments, one or more of the following may include a lubricant to facilitate smooth translation: the advancement element 26, the housing 12, and the lumen 18. In some embodiments, the lubricant may include KEMAMIDE® or another additive. In some embodiments, one or more of the following may be constructed of a lubricious material to facilitate smooth translation. In some embodiments, the lumen 18 may be vented to maintain atmospheric pressure and allow movement of air within the lumen 18 during translation of the advancement element 26 and the handle 24 in distal and/or proximal directions.

In some embodiments, the handle 24 may include grip surfaces that may facilitate bi-directional movement of the handle 24. In some embodiments, the handle 24 may be annular or extend around an entire circumference or perimeter of the housing 12. In other embodiments, the handle 24 may include a gap 62. In some embodiments, the other lumen 48 may extend through the distal end 14 of the housing 12 and the proximal end 16 of the housing 12. In some embodiments, the other lumen 48 may be aligned with the gap 62. In some embodiments, the other lumen 48 may be parallel to the lumen 18. Referring now to FIG. 1H, in some embodiments, the other lumen 48 may be disposed on top of the lumen 18 or away from skin of the patient, which may allow the user to visualize blood within the other lumen indicating placement of the catheter of the catheter assembly within the vasculature of the patient.

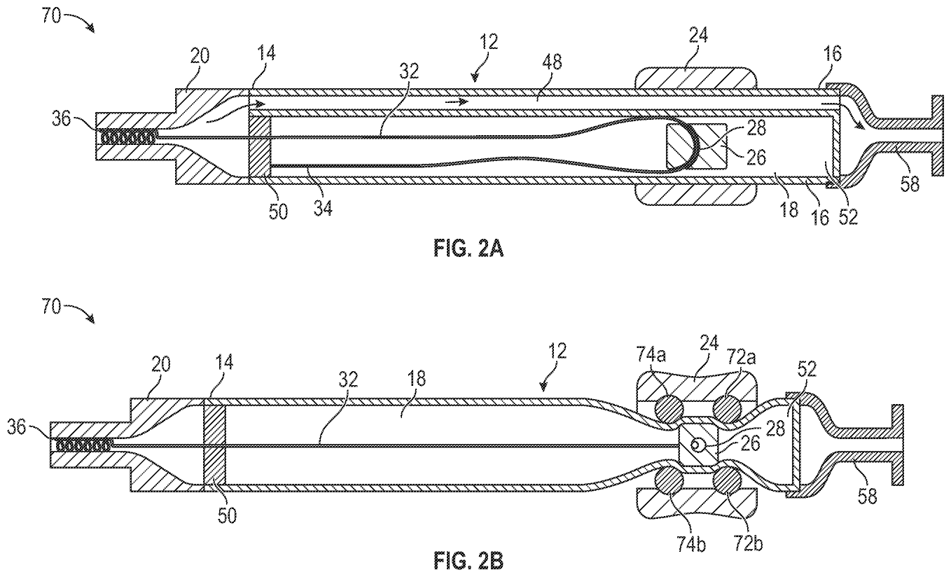

Referring now to FIGS. 2A-2B, a catheter extension set 70 is illustrated, according to some embodiments. In some embodiments, the catheter extension set 70 may be similar or identical to the catheter extension set 70 in terms of one or more features and/or operation. In some embodiments, the catheter extension set 70 may include a pair of opposing translation features 72a,72b disposed between the handle 24 and an outer surface of the housing 12. In some embodiments, the pair of opposing translation features 72a,72b may be disposed proximal to the advancement element 26 and configured to push the advancement element 26 distally. In some embodiments, in response to distal movement of the handle 24 along the outer surface of the housing 12 the first distance, the pair of opposing translation features 72a,72b may push the advancement element 26 distally within the lumen 18 the first distance, and the second end 36 of the instrument 32 may distally advance the second distance.

In some embodiments, the catheter extension set 70 may include another pair of opposing translation features 74a,74b disposed between the handle 24 and an outer surface of the housing 12. In some embodiments, the other pair of opposing translation features 74a,74b may be disposed distal to the advancement element 26 and may be configured to push the advancement element 26 proximally in response to proximal movement of the handle 24.

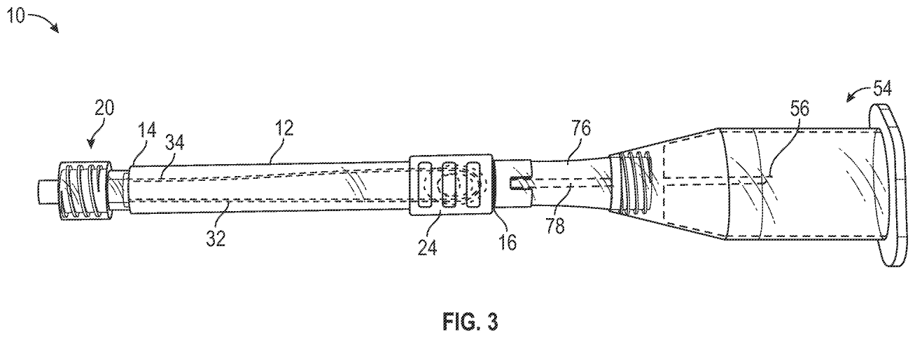

Referring now to FIG. 3, in some embodiments, the catheter extension set 10 (or the catheter extension set 70 described with respect to FIG. 2) may include a grip feature 76 proximate and proximal to the handle 24 when the instrument 32 is in a fully retracted position. In some embodiments, an outer surface of the grip feature 76 may be textured, which may facilitate gripping. For example, the outer surface of the grip feature 76 may include one or more protrusions.

In some embodiments, a distal end of the grip feature 76 may be coupled to the proximal connector 58 or the proximal end 16 of the housing 12. In some embodiments, a proximal end of the grip feature 76 may be coupled to the blood collection device 54. In some embodiments, a fluid pathway 78 may extend from the other lumen 48 through the grip feature 76 to the blood collection device 54.

In some embodiments, the user may grip the grip feature 76 with a thumb and middle finger of a hand of the user and advance the instrument 32 by placing an index finger of the hand on a top of the handle 24 and sliding the handle 24 distally. In some embodiments, the user may also slide the handle 24 proximally with the index finger on the top of the handle 24. Thus, in some embodiments, the catheter extension set 10 may be configured for operation with a single hand. In some embodiments, other gripping arrangements are also possible.

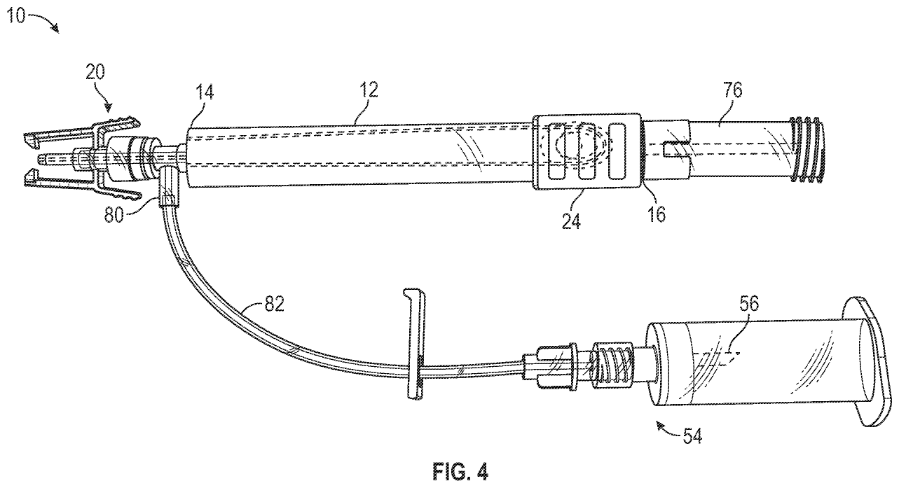

Referring now to FIG. 4, in some embodiments, the catheter extension set 10 (or the catheter extension set 70) may include a port 80 and an extension tube 82 coupled to the port 80. In some embodiments, the port 80 may be disposed distal to the elastomeric seal 50 (see, for example, FIGS. 1E and 2A-2B) and configured for fluid flow therethrough. In some embodiments, the blood collection device 54 may be coupled to the extension tube 82. In some embodiments, the blood collection device 54 (see, for example, FIG. 1A) may be coupled to the extension tube 82 via a connector 84. In some embodiments, the extension tube 82 may be integrated with the blood collection device 54 and/or the port 80. In other embodiments, as illustrated, for example, in FIG. 4, the housing 12 may not include the other lumen 48 and/or blood may flow distally through the port 80 and the extension tube 82 to the blood collection device 54.

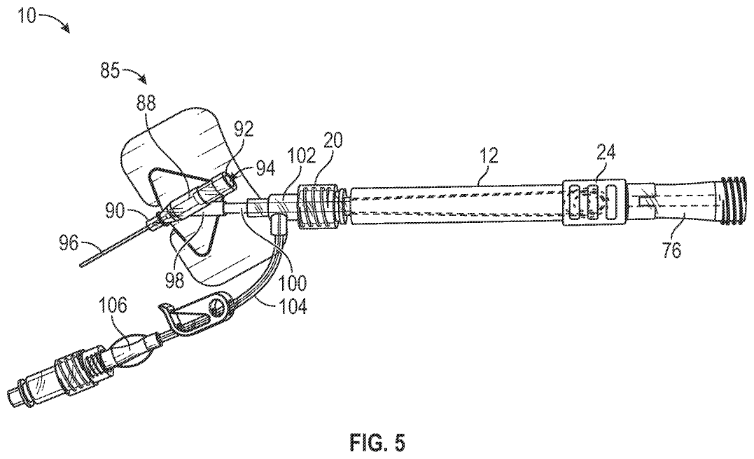

Referring now to FIG. 5, a catheter system 85 is illustrated, according to some embodiments. In some embodiments, the catheter system 85 may include a catheter assembly 86. In some embodiments, the catheter assembly 86 may include the catheter adapter 88, which may include a distal end 90, a proximal end 92, and a lumen 94 extending through the distal end 90 and the proximal end 92. In some embodiments, the catheter assembly 86 may include the catheter 96, which may extend distally from the catheter adapter 88.

In some embodiments, the catheter 96 may include a peripheral intravenous catheter, a midline catheter, or a peripherally inserted central catheter. In some embodiments, the catheter 96 may include any suitable catheter known in the art. In some embodiments, a distal tip of the catheter 96 may include one or more diffuser holes and/or may include an anti-thrombogenic coating. In some embodiments, the distal tip of the catheter 96 may be asymmetrical internally and/or externally. In some embodiments, the catheter 96 may be constructed of fluorinated ethylene propylene (FEP), polytetrafluoroethylene (PTFE), silicon, TPE, TPU, a fluorinated polymer, etc.

In some embodiments, a needle assembly (not illustrated) may be coupled to the proximal end 92 of the catheter adapter 88 and may include an introducer needle extending through the catheter 96. In some embodiments, the needle assembly may be removed from the catheter assembly 86 in response to the catheter 96 being positioned within vasculature of a patient and/or observation of flashback of blood.

In some embodiments, the catheter adapter 88 may include a side port 98 disposed between the distal end 90 and the proximal end 92 of the catheter adapter 88. In some embodiments, a distal end of an extension tube 100 may be integrated with the side port 98 and/or a proximal end of the extension tube 100 may be integrated with a connector 102. In some embodiments, the connector 102 may be coupled to the catheter extension set 10 (or the catheter extension set 70 described with respect to FIG. 2). In some embodiments, another extension tube 104 may extend from the connector 102 and may be coupled to the blood collection device (see, for example, FIG. 1A), such as, for example, via another connector 106. In some embodiments, the catheter assembly 86 and its components may vary. In some embodiments, the catheter extension set 10 (or the catheter extension set 70 described with respect to FIG. 2) may be connected to another portion of the catheter assembly 86 than illustrated in FIG. 5.

In some embodiments, in response to insertion of the catheter 96 into the vasculature of the patient, blood may flow in a fluid pathway that may extend through the catheter assembly 86 and the other lumen 48 to the blood collection device 54. In other embodiments, as illustrated in FIG. 5, the housing 12 may not include the other lumen 48 and/or blood may flow distally through the connector 102 and the other extension tube 104 to the blood collection device 54.

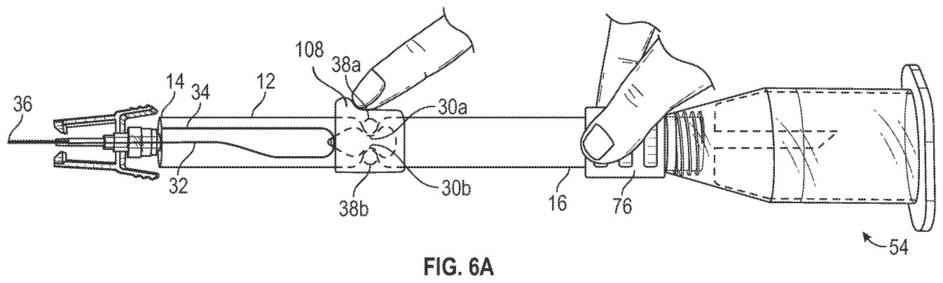

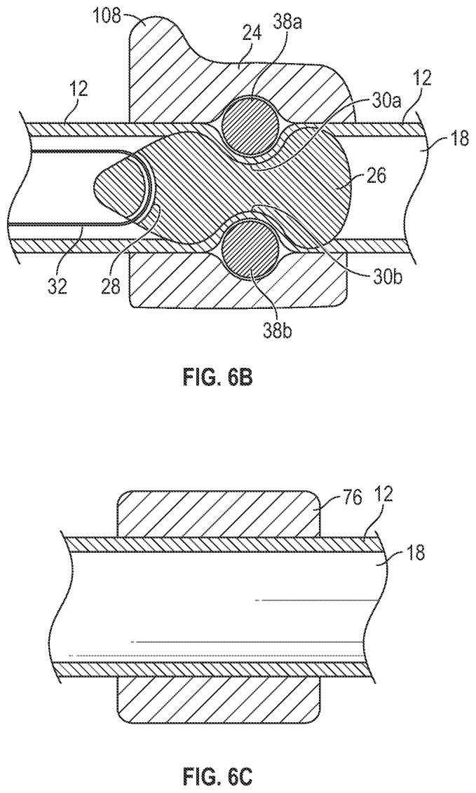

Referring now to FIG. 6A-6B, in some embodiments, the top of the handle 24 may include a protrusion 108 disposed at or towards a distal end of the handle 24. In some embodiments, the protrusion 108 may facilitate pushing of the handle 24 by the index finger or another suitable digit in the distal direction to advancement the instrument 32. In some embodiments, the thumb and the middle finger may grip the grip feature 76, as illustrated, for example, in FIG. 6A, facilitate operation with the single hand.

In some embodiments, the advancement element 26 may include multiple pockets 30. For example, the advancement element 26 may include a first pocket 30a that may oppose a second pocket 30b. In some embodiments, a first translation feature 38a may compress the housing 12 into the first pocket 30a, and a second translation feature 38b may compress the housing 12 into the second pocket 30b.

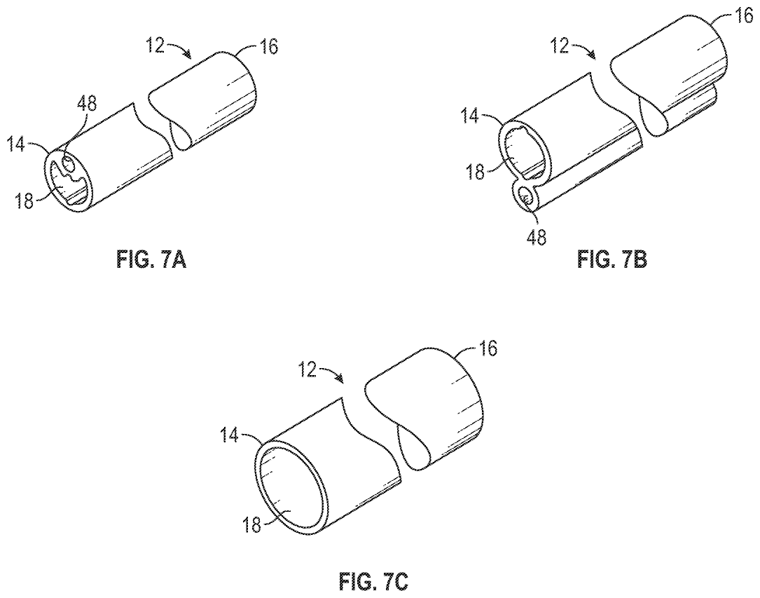

Referring now to FIG. 7A-7B, in some embodiments, an arrangement of the lumen 18 and the other lumen 48 with respect to each other may vary. In some embodiments, the lumen 18 may have a larger inner diameter than the other lumen 48. In some embodiments, the other lumen 48 may be disposed on top of the lumen 18 and further from skin of the patient, which may facilitate visualization of the other lumen 48 and blood flow therein by the user. In some embodiments, the lumen 18 and the other lumen 48 may be side by side, as illustrated, for example, in FIG. 2. In some embodiments, the lumen 18 and the other lumen 48 may be disposed in a single extension tube, as illustrated, for example, in FIGS. 7A-7B, or the lumen 18 and the other lumen 48 may be disposed in two separate tubes. Referring now to FIG. 7C, in some embodiments, the housing 12 may not include the other lumen 48 as a blood collection pathway does not extend through the housing 12 but is disposed elsewhere as illustrated in FIGS. 4-5, for example.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed is:

1. A catheter extension set, comprising:
a housing, comprising a distal end, a proximal end, and a lumen extending through the distal end of the housing;
a distal connector coupled to the distal end of the housing and configured to couple to a catheter assembly;
a handle;
an advancement element comprising a U-shaped channel and a pocket, wherein the advancement element is disposed within the lumen;
an instrument extending through the U-shaped channel, wherein the instrument comprises a fixed first end and a second end, wherein in response to distal movement of the handle a first distance, the second end is configured to advance distally a second distance, wherein the second distance is at least twice the first distance; and
a translation feature disposed between the handle and an outer surface of the housing and within the pocket such that in response to distal movement of the handle the first distance, the advancement element moves distally within the lumen the first distance and the second end of the instrument advances distally the second distance.

2. The catheter extension set of claim 1, wherein the handle does not contact the housing.

3. The catheter extension set of claim 1, wherein the housing comprises a flexible extension tube.

4. The catheter extension set of claim 1, wherein the housing further comprises another lumen, wherein the other lumen extends through the distal end of the housing and the proximal end of the housing and is configured for fluid flow therethrough.

5. The catheter extension set of claim 4, further comprising an elastomeric seal disposed within the distal connector or a distal end of the housing, wherein the instrument is configured to extend through the elastomeric seal, wherein the elastomeric seal is positioned to allow distal fluid flow into the other lumen but not the lumen.

6. The catheter extension set of claim 4, further comprising a blood collection device in fluid communication with the distal connector and the other lumen.

7. The catheter extension set of claim 6, wherein the blood collection device comprises a syringe or a sharp cannula configured to receive an evacuated blood collection tube.

8. The catheter extension set of claim 7, further comprising a proximal connector coupled to a proximal end of the housing.

9. The catheter extension set of claim 8, further comprising a blood collection device coupled to the proximal connector.

10. The catheter extension set of claim 1, wherein the handle comprises a gap, wherein the housing further comprises another lumen, wherein the other lumen extends through the distal end of the housing and the proximal end of the housing and is configured for fluid flow therethrough, wherein the other lumen is aligned with the gap, wherein the other lumen is disposed on top of the lumen.

11. The catheter extension set of claim 1, wherein a proximal end of the lumen is closed.

12. The catheter extension set of claim 1, further comprising an additional translation feature disposed on an opposite side of the housing from the translation feature and between the housing and the handle.

13. The catheter extension set of claim 1, further comprising an elastomeric seal disposed within the distal connector or a distal end of the housing, wherein the instrument is configured to extend through the elastomeric seal, further comprising a port and an extension tube coupled to the port, wherein the port is disposed distal to the elastomeric seal and configured for fluid flow therethrough.

14. The catheter extension set of claim 1, further comprising a grip feature proximate and proximal to the handle when the instrument is in a fully retracted position, wherein an outer surface of the grip feature is textured.

15. A catheter extension set, comprising:
a housing, comprising a distal end, a proximal end, and a lumen extending through the distal end of the housing;
a distal connector coupled to the distal end of the housing and configured to couple to a catheter assembly;
a handle;
an advancement element comprising a U-shaped channel, wherein the advancement element is disposed within the lumen;
an instrument extending through the U-shaped channel, wherein the instrument comprises a fixed first end and a second end, wherein in response to distal movement of the handle a first distance, the second end is configured to advance distally a second distance, wherein the second distance is at least twice the first distance; and
a pair of opposing translation features disposed between the handle and an outer surface of the housing, wherein the pair of opposing translation features is disposed proximal to the advancement element and configured to push the advancement element distally, wherein in response to distal movement of the handle along the outer surface of the housing the first distance, the pair of opposing translation features pushes the advancement element distally within the lumen the first distance, and the second end of the instrument advances distally the second distance.

16. The catheter extension set of claim 15, further comprising another pair of opposing translation features disposed between the handle and an outer surface of the housing, wherein the other pair of opposing translation features are disposed distal to the advancement element and are configured to push the advancement element proximally in response to proximal movement of the handle.

17. The catheter extension set of claim 15, wherein the housing further comprises another lumen, wherein the other lumen extends through the distal end of the housing and the proximal end of the housing and is configured for fluid flow therethrough.

18. The catheter extension set of claim 17, further comprising a blood collection device in fluid communication with the distal connector and the other lumen.

19. A method of blood collection, comprising:
inserting a catheter of a catheter assembly into vasculature of a patient, wherein the catheter assembly comprises:
a catheter adapter, comprising a distal end, a proximal end, and a lumen extending through the distal end of the catheter adapter and the proximal end of the catheter adapter; and
the catheter extending from the distal end of the catheter adapter;
coupling a catheter extension set to the catheter assembly, wherein the catheter extension set comprises:
a housing, comprising a distal end, a proximal end, and a lumen extending through the distal end of the housing;
a distal connector coupled to the distal end of the housing and configured to couple to a catheter assembly;
a handle;
an advancement element comprising a U-shaped channel and a pocket, wherein the advancement element is disposed within the lumen;
an instrument extending through the U-shaped channel, wherein the instrument comprises a fixed first end and a second end, wherein in response to the handle advancing distally a first distance, the second end of the instrument is advance distally a second distance in response to distal movement of the handle a second distance, wherein the second distance is at least twice the first distance; and
a translation feature disposed between the handle and an outer surface of the housing and within the pocket; and
moving the handle distally the first distance, wherein in response to moving the handle distally the first distance, the second end of the instrument advances into or through the catheter.

20. The method of claim 19, wherein the housing further comprises another lumen, wherein the other lumen extends through the distal end of the housing and the proximal end of the housing and is configured for fluid flow therethrough, wherein the method further comprises:
moving the handle proximally the first distance, wherein in response to moving the handle proximally the first distance, the second end of the instrument is retracted into the catheter extension set; and
collecting blood through the other lumen of the housing.
